(19) United States
(12) Patent Application Publication
(10) Pub. No.: US 2022/0000601 A1
(43) Pub. Date: Jan. 6, 2022

(54) METHODS, SYSTEMS, AND DEVICES FOR EMBOLIC PROTECTION

(71) Applicant: BALEEN MEDICAL LLC, 94301, CA (US)

(72) Inventors: Sebastian KHAIRKHAHAN, Palo Alto, CA (US); Alex KHAIRKHAHAN, Palo Alto, CA (US)

(21) Appl. No.: 17/294,348

(22) PCT Filed: Nov. 15, 2019
(86) PCT No.: PCT/US2019/061739

§ 371 Date: May 14, 2021

Related U.S. Application Data
(60) Provisional application No. 62/792,362, filed on Jan. 14, 2019.
(60) Provisional application No. 62/767,860, filed on Nov. 15, 2018.

(51) Int. Cl. A61F 2/01

(52) U.S. Cl. CPC A61F 2/013; A61F 2250/0098; A61F 2002/016; A61F 2/011

(57) ABSTRACT

Embodiments of the present disclosure include, for example, an embolic protection system (EPS) including an inner-body having a body diameter and a distal section, and an expandable filter arranged on or adjacent at least the distal section of the inner-body. The filter is configured to include a plurality of pores, sized to allow the flow of the blood with limited interruption and capture of emboli greater than the pore size. Such embodiments may also include an expandable introducer sheath/sleeve having a sheath diameter configured to accommodate the inner-body and filter, including the distal portion, when unexpanded, as well as a tear-away (TA) sleeve having a sleeve diameter configured to accommodate the introducer sheath containing the inner-body and filter when unexpanded.

### RELATED APPLICATIONS

The subject application claims benefit of and priority to U.S. provisional patent application No. 62/767,860, filed Nov. 15, 2018, and entitled, “SYSTEMS, APPARATUSES, DEVICES AND METHODS FOR EMBOLIC PROTECTION,” and 62/792,362, filed Jan. 14, 2019, entitled, “METHODS, SYSTEMS, AND DEVICES FOR EMBOLIC PROTECTION,” both disclosures of which are herein incorporated by reference in their entireties.

### BACKGROUND OF THE DISCLOSURE

In the past decade, major developments have taken place in catheter-based treatments for structural heart diseases such Transcatheter Aortic Valve Replacement (TAVR) and Transcatheter Mitral Valve replacement (TMVR). Cerebral embolism is a known complication of such procedures where embolic particles which may include thrombus, atheroma, and lipids may dislodge during the implantation procedure, enter the blood stream embolizing in the brain and other vital organs. Cerebra embolism could result in serious neurological deficits, stroke, and even death. Furthermore, embolism in vital organs such as kidneys could severely compromise the function of these organs resulting in hospitalization, diminished quality of life and in some cases death. Therefore, preventing the embolism in the brain and other vital organs could hugely benefit the patient by improving the outcome of these procedures.

### SUMMARY OF AT LEAST SOME EMBODIMENTS OF THE DISCLOSURE

Embodiments of the present disclosure are directed to methods, systems and devices for embolic protection, and more specifically, to methods, systems, and devices for embolic protection for surgical systems and methods including, for example, delivery/implanting systems as well as methods for delivering or implanting prosthetic heart valves into the heart, or performing a cardiac or blood vessel procedure, where capturing or otherwise trapping emboli dislodged or created during the procedure is necessary so as to prevent complications associated therewith (e.g., strokes).

Accordingly, in some embodiments, an embolic protection system (EPS) is provided and comprises or otherwise includes an inner-body having a body diameter and a distal section, and an expandable filter arranged on or adjacent at least the distal section of the inner-body. The filter is configured to include a plurality of pores sized between, in some embodiments, approximately 0.100 mm-1.000 mm, to allow the flow of the blood with limited interruption and capture of emboli greater than the pore size. Such embodiments may further include a radiopaque marker arranged on one or more areas of at least one of the filter and distal section of the inner-body, where the marker may comprise at least one strand of at least one of platinum and gold included with the filter, and/or a coating of at least one of platinum and gold.

In addition, the above-noted embodiments may further include an expandable introducer sheath (IS), which may also be referred to as a sleeve, having a sheath diameter configured to accommodate the inner-body and filter, including the distal portion, when unexpanded. In some embodiments, the introducer sheath may include a wall having a thickness, in some embodiments, of between approximately 0.025 to 0.250 mm. The introducer sheath may further include an introducer hub arranged on a proximal end thereof, and a flush port.

The above-noted embodiments may further include a tear-away (TA) sleeve having a sleeve diameter configured to accommodate the introducer sheath containing the inner-body and filter when unexpanded, and a dilator having a dilator diameter configured fit within the introducer sheath and within the body diameter, where the dilator may include a conical distal tip and a proximal dilator hub.

In some of at least the above-noted embodiments:
the TA sleeve includes at least one pull-tab at a proximal end and is configured to tear or otherwise separate along at least a portion of its length beginning at the proximal end at least initially via the at least one pull-tab as the TA sleeve is pulled or otherwise directed proximally prior to expansion of at least the filter;
at least the filter expands as it is distally exposed by the withdrawal of the introducer sheath in the proximal direction, such that the introducer sheath proximally folds upon itself in an accordion-like fashion;
at least a portion of the filter and/or distal section of the inner-body is configured to expand to a size to apply a force against an inner wall of a blood vessel upon which the filter and/or distal section of the inner-body is positioned;
the introducer sheath is configured to expand over at least a portion of its length between approximately 10 cm and 30 cm;

and/or
at least the expanded filter is configured for recapture after expansion by the distal end of introducer sheath upon distal movement of the introducer sheath.

In some embodiments, an embolic protection system (EPS) is provided and comprises an expandable filter including a plurality of pores sized between, in some embodiments, approximately 0.100 mm-1.000 mm, to allow the flow of the blood with limited interruption and capture of emboli greater than the pore size, an introducer sheath having a sheath diameter configured to accommodate at least the filter when unexpanded, where the introducer sheath includes a wall having a thickness of between, in some embodiments, approximately 0.025 to 0.250 mm, and a tear-away (TA) sleeve having a sleeve diameter configured to accommodate the introducer sheath containing at least filter when unexpanded. In such embodiments, the TA sleeve is configured to tear or otherwise separate along at least a portion of its length beginning at the proximal end as the TA sleeve is pulled or otherwise directed proximally prior to expansion of the filter, and the filter expands as it is distally exposed by the withdrawal of the introducer sheath in the proximal direction, such that the introducer sheath proximally folds upon itself.

Such embodiments may include one and/or another (and in some embodiments, a plurality of, and in further embodiments, all of) of the following features, clarifications, structures, or functionality (as applicable):
at least the expanded filter is configured for recapture after expansion by the distal end of the introducer sheath upon distal movement of the introducer sheath;
the introducer sheath proximally folds upon itself in an accordion-like fashion;
an inner-body having a body diameter and a distal section;
the filter is arranged on or adjacent at least the distal section of the inner-body;
a radiopaque marker arranged on one or more areas of at least the filter;
   the marker may comprise at least one strand of at least one of platinum and gold included with the filter, and/or a coating of at least one of platinum and gold;
a radiopaque marker arranged on one or more areas of at least one of the filter and distal section of the inner-body;
   the marker may comprise at least one strand of at least one of platinum and gold included with the filter, and/or a coating of at least one of platinum and gold;
the introducer sheath diameter can be configured to accommodate the inner-body and filter, including the distal portion, when unexpanded;
the introducer sheath includes at least one of: a wall which may have a thickness of between approximately 0.025 to 0.250 mm, an introducer hub arranged on a proximal end thereof, and a flush port;
the sleeve diameter of the TA may be configured to accommodate the introducer sheath containing the inner-body and filter when unexpanded;
a dilator having a dilator diameter configured fit within the IS, where the dilator may include at least one of a conical distal tip and a proximal dilator hub;
a dilator having a dilator diameter configured fit within the IS, and within the body diameter, where the dilator may include at least one of a conical distal tip and a proximal dilator hub;
the TA sleeve may include at least one pull-tab at a proximal end;
the TA may be configured to tear or otherwise separate along at least a portion of its length beginning at the proximal end, and, in some embodiments, such tearing occurs at least initially via the at least one pull-tab as the TA sleeve is pulled or otherwise directed proximally prior to expansion of at least the filter;
at least a portion of the filter and/or distal section of the inner-body may be configured to expand to a size to apply a force against an inner wall of a blood vessel upon which the filter and/or distal section of the inner-body is positioned;
at least a portion of the length of the introducer sheath is configured to expand, where the expansion may be, in some embodiments, between approximately 10 cm and 30 cm;
a first housing configured to substantially encase, and in some embodiments, encase and seal, a proximal end of the TA sleeve, where the first housing may comprise a guide lumen;

and
a second housing for encasing the first housing, and in some embodiments, encasing and sealing the first housing, where the second housing comprises a guide lumen.

In some embodiments, an embolic protection introducer sheath is provided and comprises a sleeve having a sheath diameter configured to accommodate at least an expandable filter when unexpanded. The introducer sheath may include a wall having a thickness of between approximately 0.025 to 0.250 mm, and the filter may be configured to expand as it is distally exposed by movement of the introducer sheath in the proximal direction, such that the introducer sheath proximally folds upon itself. In some embodiments, the introducer sheath is configured to recapture at least the expanded filter upon movement of the introducer sheath in a distal direction.

In such embodiments, the introducer sheath proximally is configured to fold-up upon itself in an accordion-like fashion, and/or the introducer sheath is configured to expand over at least a portion of its length between, in some embodiments, approximately 10 cm and 30 cm.

In some embodiments, an embolic protection method (EPM) is provided and comprises: optionally providing an embolic protection system (EPS) according to any of the disclosed system and/or device embodiments, directing an/the EPS to a surgical location in a blood vessel or organ, pulling or otherwise moving a/the tear-away (TA) sleeve in a proximal direction, tearing or otherwise separating the TA sleeve along at least a portion of its length beginning at the proximal end as the TA sleeve is pulled or otherwise moved in the proximal direction, and pulling or otherwise directing an/the introducer sheath in a proximal direction to expose a/the filter, such that: as the filter is exposed it expands, and the introducer sheath folds upon itself. In some embodiments, at least a portion of the filter and/or a distal section of the inner-body is configured to expand to a size to apply a force against an inner wall of the blood vessel or organ upon which the filter and/or distal section of the inner-body is positioned. Moreover, in some embodiments, the method further includes recapturing the expanded filter by moving the introducer sheath distally.

Such embodiments may include one and/or another (and in some embodiments, a plurality of, and in further embodiments, all of) of the following steps, features, clarifications, structures, or functionality (as applicable):
performing a surgical procedure, such that, the filter is configured to capture emboli produced and/or dislodged by the procedure;
the procedure is selected from the group consisting of: transvascular or transapical prosthetic heart valve implantation or native heart valve repair;
expanding at least a portion of the length of the IS;

and
the at least a portion of the length of the introducer sheath is expanded between approximately 10 cm and 30 cm.

These and other embodiments, components, materials, steps, and advantages and objects thereof will become even more apparent with reference to the detailed description which follows, and reference to the associated figures, a brief description of which is provided below.

### BRIEF DESCRIPTION OF THE FIGURES

FIG. 1 is a side view of an inner-body member of a embolic protection system or device according to some embodiments of the disclosure.

FIG. 2 is a side-view of a filter element according to some embodiments, which may be placed over or otherwise adjacent the inner-body of FIG. 1, according to some embodiments of the disclosure;

FIG. 3A is a side-view of a filter element placed over or otherwise adjacent the inner-body of FIG. 1, forming an assembly, according to some embodiments of the disclosure;

FIG. 3B is a perspective view of a filter and inner-body assembly, according to some embodiments of the disclosure;

FIG. 3C is another perspective view of a filter and inner-body assembly, according to some embodiments of the disclosure;

FIG. 3D is a side view of a filter and inner-body assembly, according to some embodiments of the disclosure;

FIG. 4 illustrates a side view of a tear-away (TA) sleeve, according to some embodiments of the disclosure;

FIG. 5 is a side view of an expandable introducer sheath/sleeve assembly, according to some embodiments of the disclosure;

FIG. 6 is a side view of a tear-away introducer sheath according to some embodiments of the disclosure;

FIGS. 7A-B are side views of a first clamshell for encasing and/or sealing a first/handle portion of the tear-away introducer sheath of FIG. 6, according to some embodiments, along with a second clamshell guide lumen for encasing and/or sealing the first clamshell, in both disassembled (FIG. 7A), and assembled (FIG. 7B), according to some embodiments;

FIG. 8A is a side view of half of the second clamshell encasing and/or sealing the half first clamshell, as well as the handle portion being housed in half of the first clamshell, according to some embodiments;

FIG. 8B is a side view of the full second clamshell encasing and/or sealing the first clamshell, as well as the handle portion being housed in the first clamshell, according to some embodiments;

FIG. 9 is a side view of a dilator for use with the system and/or device according to some embodiments of the disclosure;

FIG. 10 is a side view of an assembly of the tear-away sleeve and expandable introducer sheath assembly, according to some embodiments of the disclosure;

FIG. 11 is a side view of an assembly, according to some embodiments, including the dilator, the expandable introducer sheath/sleeve assembly, and tear-away sleeve;

FIG. 12A is a side view of the assembly prior to use, according to some embodiments of the disclosure;

FIG. 12B is a side view of the assembly upon to use, according to some embodiments of the disclosure;

FIGS. 13A-B are side views of the introducer sleeve, illustrating sleeve functionality, according to some embodiments of the disclosure; and

FIGS. 14A-C illustrate overviews of use of an embolic protection system according to some embodiments of the disclosure, illustrating methodology for performing an exemplary procedure (e.g., cardiac, e.g., valve repair or implantation), according to some embodiments.

### FURTHER DETAILS OF AT LEAST SOME EMBODIMENTS OF THE DISCLOSURE

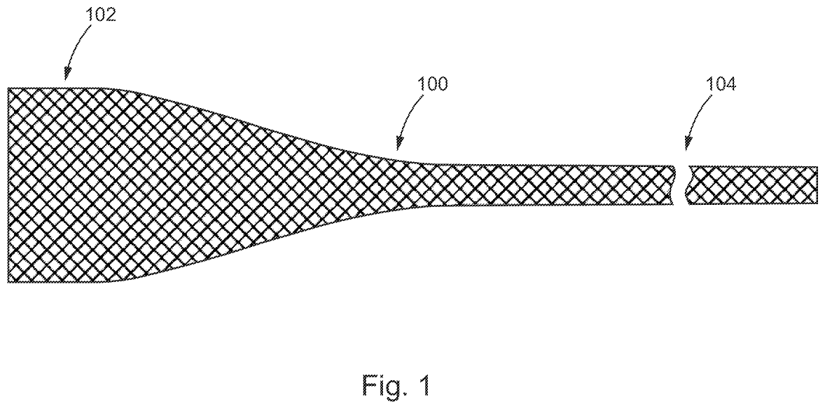

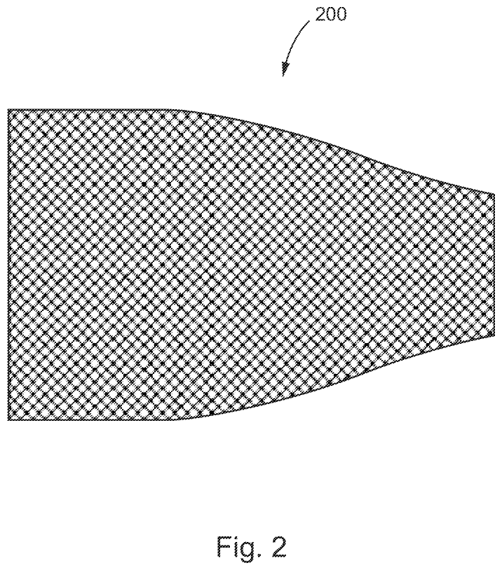

FIG. 1 is a side view of an inner-body member 100 of an embolic protection system or device according to some embodiments. The inner-body 100, in some embodiments, can be a braided material, and can be made of Nitinol, stainless steel, and/or polymeric materials (whether braided material or otherwise). A distal section 102 can be configured with an expansion capability and/or shape (e.g., a bell shape, via, e.g., heat setting) so as to achieve a diameter larger that the diameter of a blood vessel/tissue where the inner-body or system is used (e.g., an aorta), and a tubular proximal section 104. This functionality may be included so as to provide a radial outward force to push edges (at least) of the inner-body 100, and/or a filter (e.g., see FIG. 2) arranged adjacent the inner-body 100, against the inner wall of a blood vessel (or other tissue).

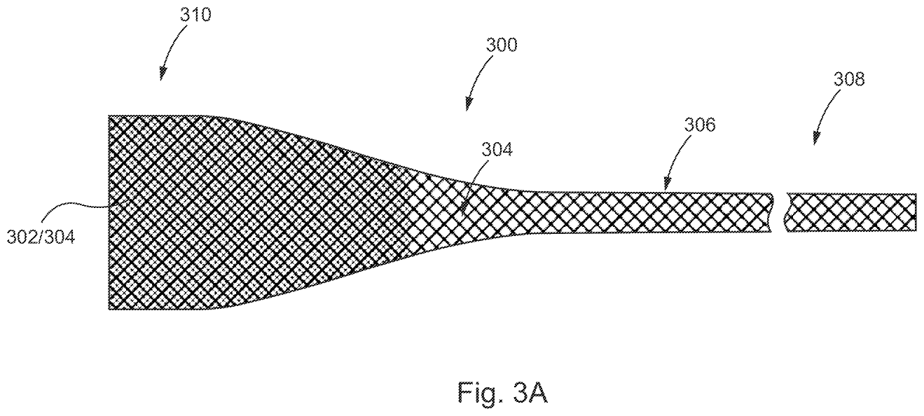

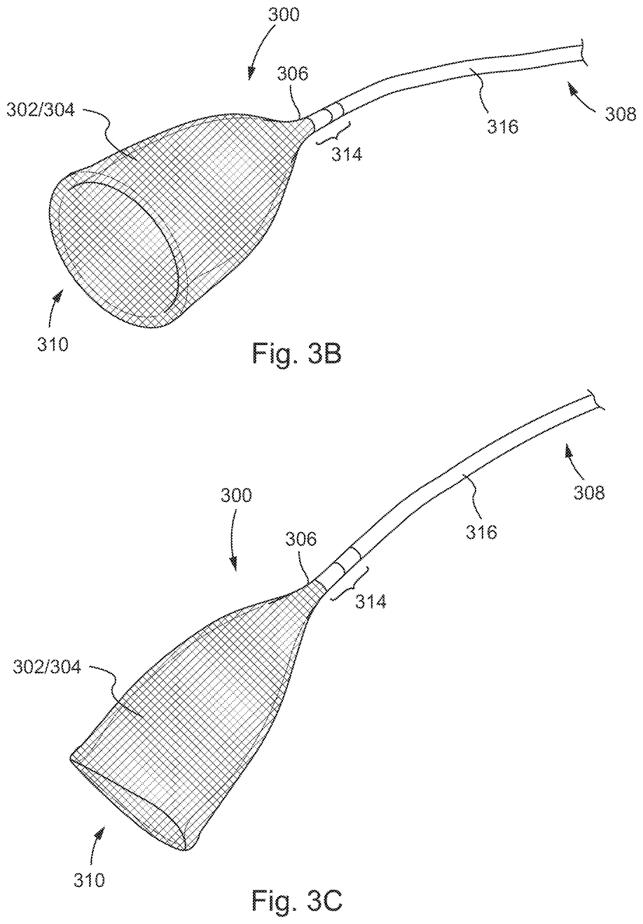

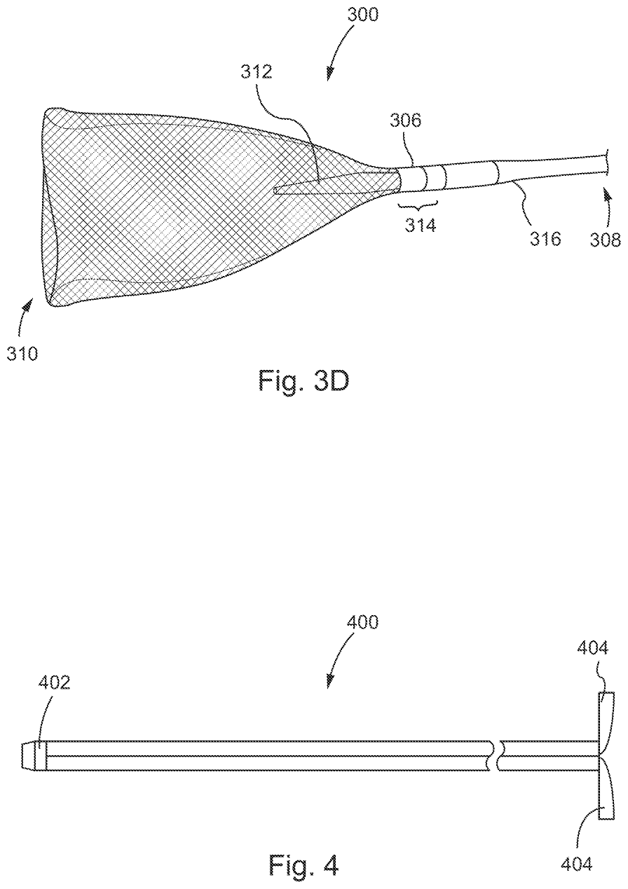

FIG. 2 is a side-view of a filter element (as referred to above) 200 shown in an expanded configuration, according to some embodiments, which can be in the form of a bell shape (similar to that of the bell shape of the inner-body illustrated in FIG. 1). Such a filter element 200, according to some embodiments, may be placed over, within, or otherwise adjacent the inner-body (e.g., see FIGS. 3A-D), and in particular, positioned (in some embodiments) with the distal portion of the inner-body, forming an assembly (e.g., see FIG. 3A). The filter 200 may also be configured as braided element, and may be made from Nitinol, stainless steel, and/or polymeric materials (whether braided material or otherwise). The pore size of the filter may be configured to allow the flow of the blood with limited interruption, but also enabling the capture of emboli and debris of a particular size (dictated by the pore size) by the filter. It will be appreciated, that in some embodiments, the distal end of the inner-body may be configured to inherently perform as a filter, and thus, a separate, distally mounted filter may be unnecessary.

FIGS. 3A-D illustrate various views of an assembly 300 of the filter 302 and inner-body 304. Further to the tubular portion 306 of the inner-body 304, in some embodiments, this allows procedures to be conducted through the tubular portion, while the assembly 300 of the filter and inner-body function to filter any debris from the procedure. The proximal portion 308 of the inner-body can be configured to form a tubular body (which may also be referred to as a shaft), which is expandable (according to some embodiments), for example, the diameter can expand between 3 mm and 10 mm. The diameter of the tubular portion can be configured to accommodate medical implants (e.g., prosthetic heart valves, surgical equipment, and the like). Such is disclosed in FIG. 3D, showing that at or near the distal end of the assembly 300, an implant 312 is shown protruding from a neck 314 are within the assembly. Thus, in some embodiments, a treatment device/implant can be delivered through the expandable proximal shaft first, and then through the expandable joint/neck/adaptor, and finally, through the filter area.

It is also worth noting that, in some embodiments, the inner-body and filter element may comprise one element, which is affixed at a neck portion (e.g., 314), via any manner familiar to one of skill in the art, to a catheter 316 (portion 316 can be referred to as the inner-body tubular portion or a separate catheter portion joined to the inner-body/filter assembly). The catheter/guide-lumen portion 316 may, in some embodiments, comprise a mesh, similar to that of the inner-body (and/or filter). In some embodiments, the neck portion 314 may be an adaptor or joiner to adapt/join the catheter 316 to the inner-body/filter assembly. This adaptor/joint can also be configured to be expandable.

In some embodiments, the filter element can comprise a portion of the inner-body, or otherwise be constructed of the same material (and the same or similar configuration). For example, in some embodiments, the distal end of the inner-body can be folded back upon itself so as to form a closer spacing of the mesh. In some embodiments, the entire length (from proximal to distal, or substantially thereto) of the inner-body can be multi-layered so as to form a closer spacing of mesh along the entire length (or substantially the entire length).

In some embodiments, one or more platinum or gold wires (not shown) can be included (e.g., woven) on or proximate to a distal edge of the filter (and/or the inner-body) to provide, for example, a radiopaque marker for placement of the filter under fluoroscopy. Alternatively, a portion of the distal end of the filter may be coated with gold (e.g., via vapor deposition). The filter and/or the braid may also be coated with anti-thrombogenic materials such as Heparin (for example).

FIG. 4 illustrates a side view of a tear-away (TA) sleeve 400, according to some embodiments of the disclosure. The TA sleeve may include a radiopaque marker 402 (shown below distally located, for example), and at least one (and as illustrated, according to some embodiments, two) tearing “wings” or pull-tabs 404, configured for gripping by a medical professional (or in some embodiments, another device), so as to aid in tearing the sleeve 400 in at least two (2) portions (e.g., two halves), longitudinally along at least a portion of its length in some embodiments, or substantially its entire length (according to some embodiments).

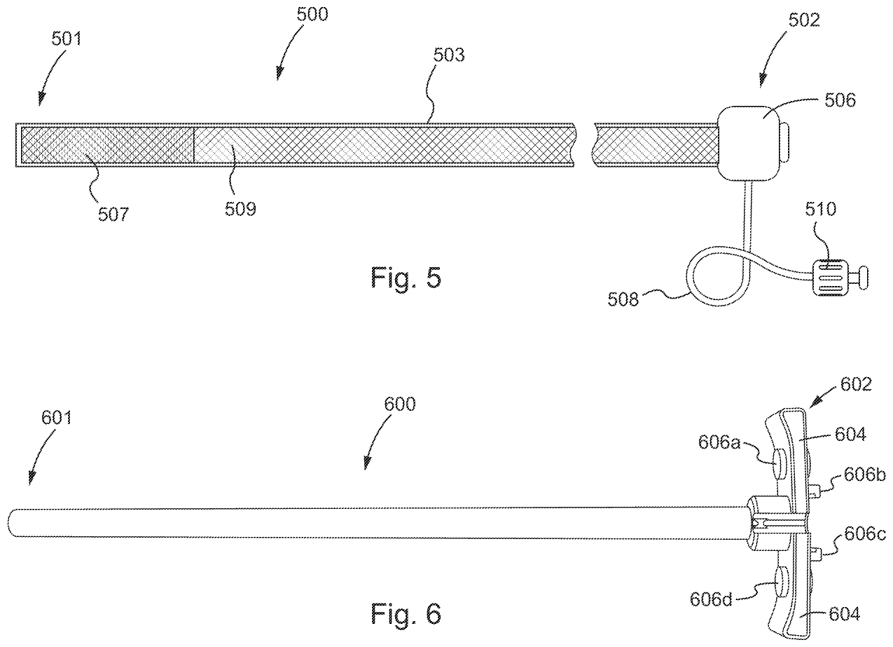

FIG. 5 is a side view of an assembly according to some embodiments, depicting an expandable introducer sheath/sleeve assembly 500, with distal portion 501 and proximal portion 502. In some embodiments, the assembly includes an expandable introducer sheath/sleeve (IS) 503, having a wall thickness, in some embodiments, between approximately 0.025 and 0.250 mm, and in some embodiments, approximately 0.050 mm. The introducer sheath 503 may be configured to cover the filter 507 and braided inner-body 509 for a smaller introduction/delivery profile for delivery into a blood vessel or organ (for example). The phrase “introducer sheath” and “introducer sleeve”, may be referred to interchangeably throughout the disclosure. Assembly 500 also includes an introducer hub 506, which can include functionality for connecting a flush tube 508 and corresponding port 510, for supplying fluids for a medical procedure.

FIG. 6 is another example of a tear-away introducer sheath 600, including a distal portion 601 and proximal portion 602, including wings/pull-tabs 604. The pull-tabs 604, in some embodiments, includes features 606a-d for connection with other elements or interlocking thereto.

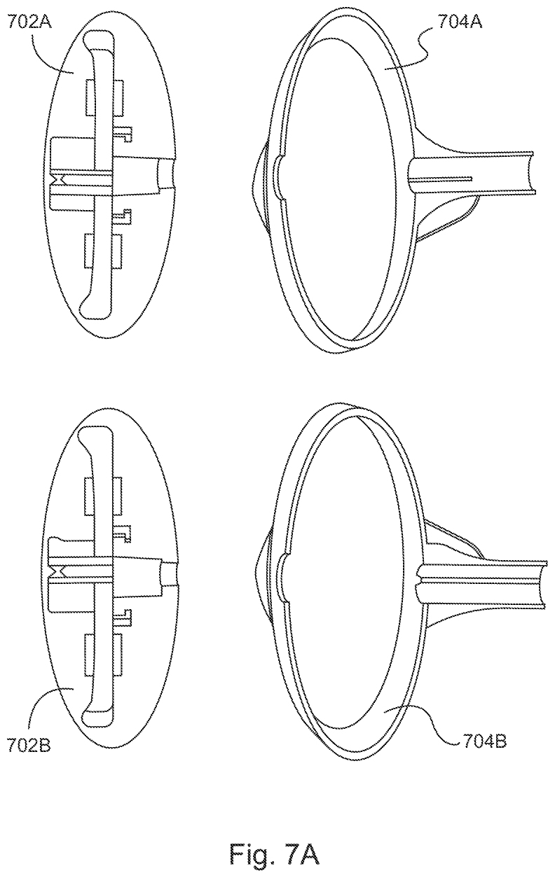

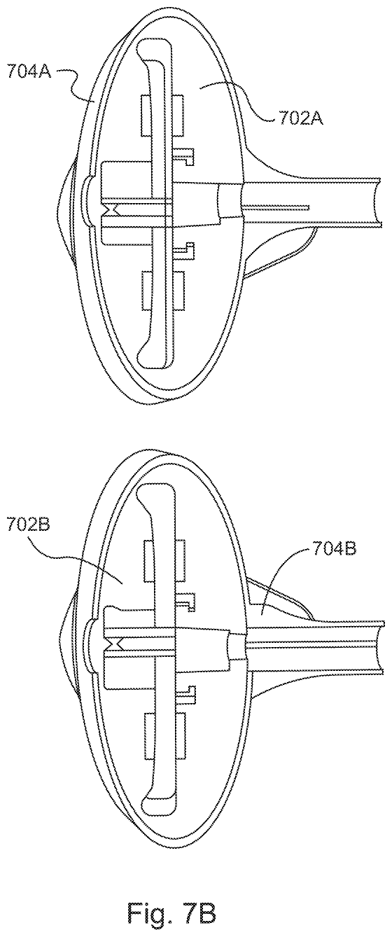

FIGS. 7A-B are side views, both disassembled and assembled, of a first clamshell housing 702A, B, which in some embodiments can be molded in a form to match or closely match that of the corresponding pull-tab portion of the introducer sleeve (see ref. no. 600, FIG. 6). The first clamshell can be further encased and/or sealed (according to some embodiments), by a second clamshell housing 704A, B, which when assembled a portion of which forms a guide lumen (e.g., see description above, with respect to some embodiments).

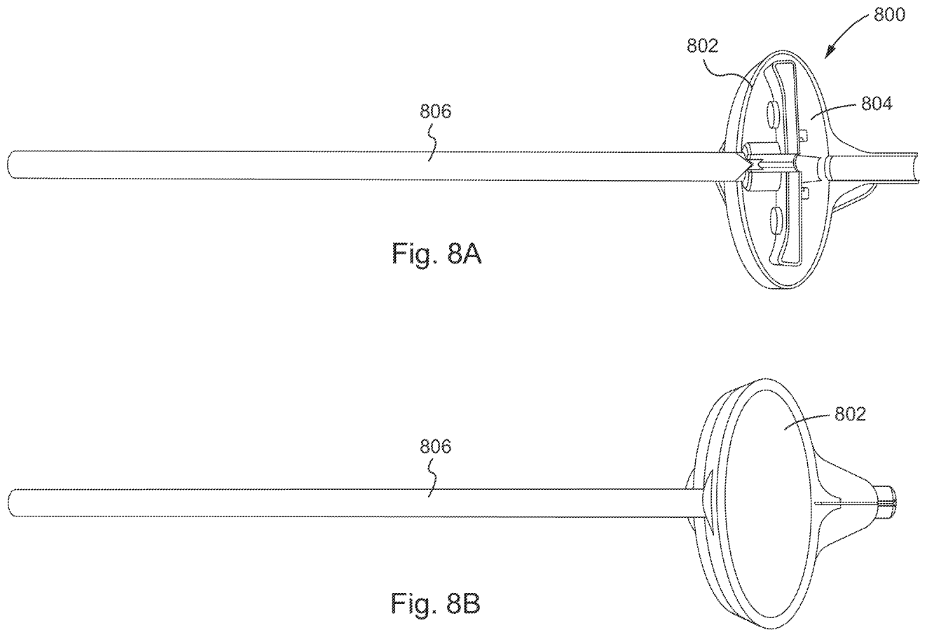

FIGS. 8A-B are side views of assembled halves 800 (FIG. 8A “half”, FIG. 8B “complete), illustrating the second clamshell 802 encasing and/or sealing the first clamshell 804, as well as the pull-tab portion of the introducer sleeve 806. The halves of the first and/or second clamshells housings, according to some embodiments, are configured in parts/portions/halves, so that they can be removable to enable the TA sleeve to “tear apart”.

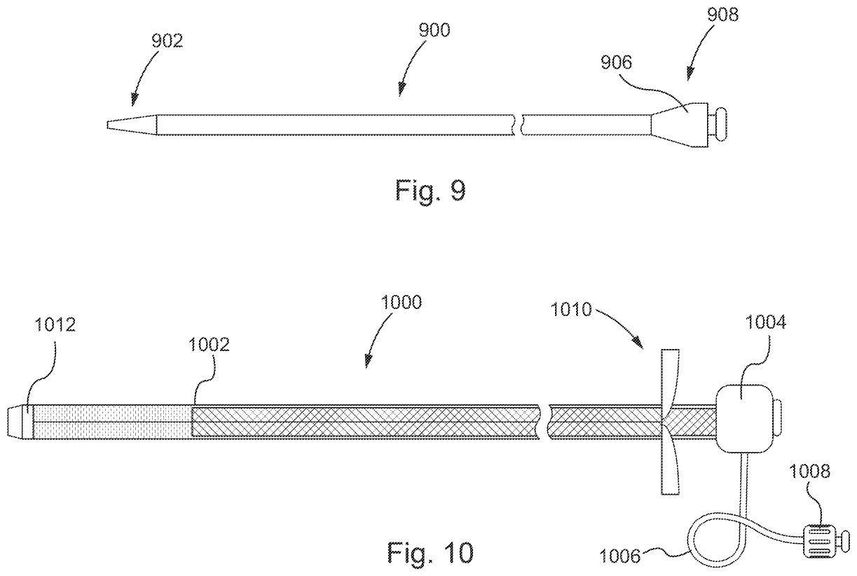

FIG. 9 illustrates is a side view of a dilator 900, with a distal end tip 902, for use with the system and/or device according to some embodiments of the disclosure, and dilator hub 906 arranged on a proximal end 908.

FIG. 10 is a side view of an assembly of the tear-away sleeve 1002, expandable introducer sheath assembly 1000 (including the inner-body and filter), according to some embodiments of the disclosure (see also, FIGS. 1-5, for example). Also shown is an introducer hub 1004, flush tube 1006, flush port 1008, TA sleeve 1010, and TA sleeve radiopaque marker 1012.

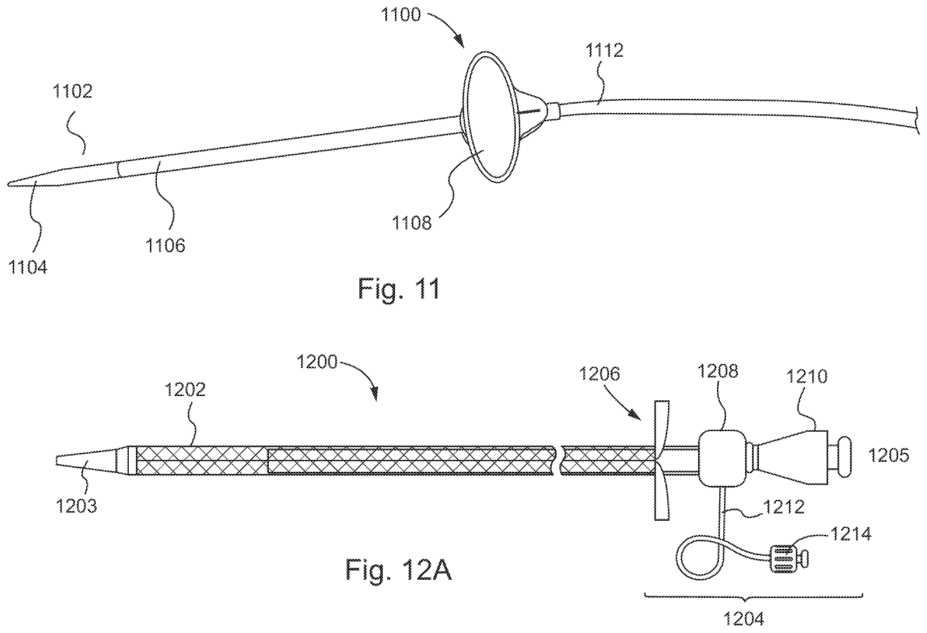

FIG. 11 illustrates a complete embolic protection system (EPS) 1100, according to some embodiments, including an inner-body having a body diameter and a distal section 1102, and an expandable filter arranged on or adjacent at least the distal section of the inner-body (not shown). Also shown, is the distal tip 1104 of the dilator 1106 component, assembled halves 1108 of the two clamshells (only guide lumen 1108 shown), as well as the tubular portion 1112 of system 1100, extending past the guide lumen 1108. At the proximal end, the shaft can be made, in some embodiments, from braided materials (e.g., polymer, nitinol), and may also be covered by a thin sleeve.

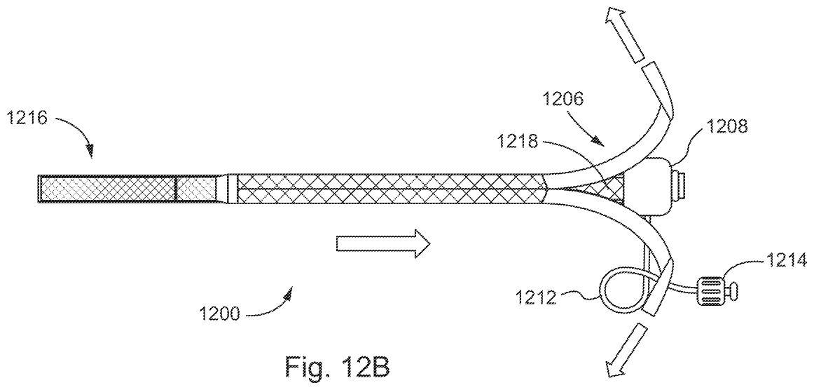

Similarly, FIG. 12A illustrates a side view of an assembly for an EPS 1200, according to some embodiments, including the dilator 1202 and distal dilator tip 1203, the expandable introducer sheath/sleeve assembly 1204, tear-away sleeve 1206, and located at the proximal end 1205, introducer hub 1208, with dilator hub 1210 and flush tube 1212 and port 1214. FIG. 12B illustrates a side view of the EPS 1200, according to some embodiments, illustrating the tearing-apart of the tear-away sleeve 1206. Specifically, as the tear-away sleeve 1206 is pulled or otherwise moved in a proximal direction, tearing or otherwise separating the TA sleeve 1206 occurs along at least a portion of its length beginning at the proximal end 1201 as the TA sleeve 1206 is pulled or otherwise moved in the proximal direction. In some embodiments, this may occur prior to at least one of the filter 1216 and the braided inner body 1218 expanding.

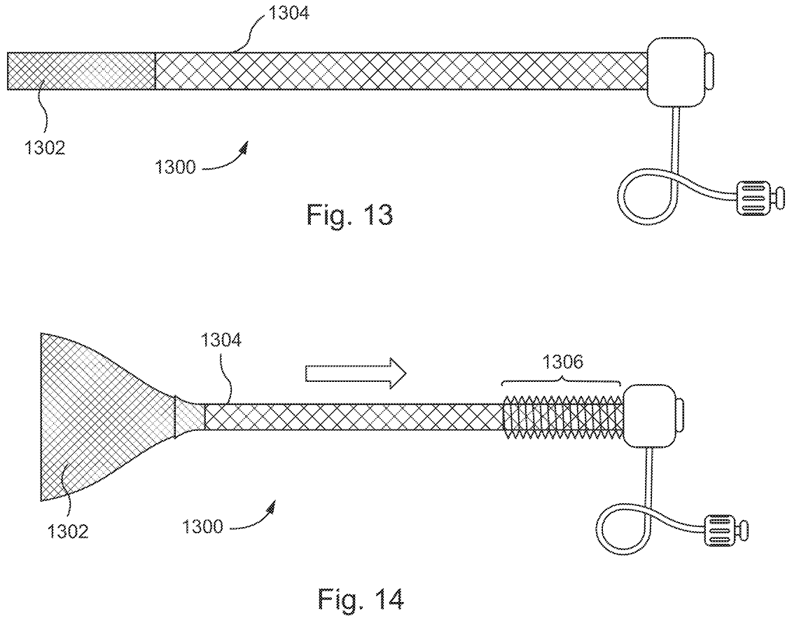

FIG. 13A-B illustrate side views of at least some of the functionality of the introducer sheath 1300, illustrating how the filter 1302 (and/or distal end of the braided inner-body, in some embodiments) is exposed as the expandable sleeve 1304 is pulled back. In some embodiments, this results in the proximal section of the introducer sheath being collapsed in an, for example, accordion fashion 1306 on itself over the proximal braided section (for example). Such “Accordion” functionality includes any ability, shape, and/or form of the introducer sheath to collapse upon itself.

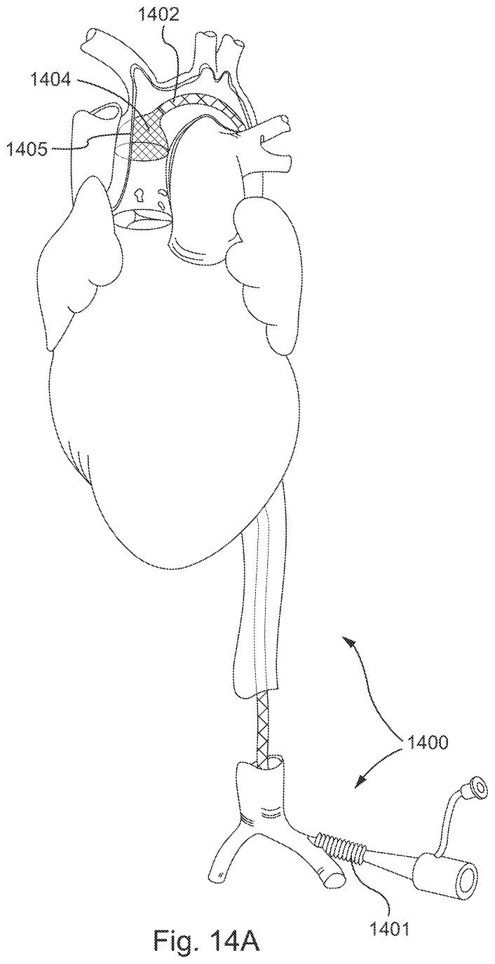

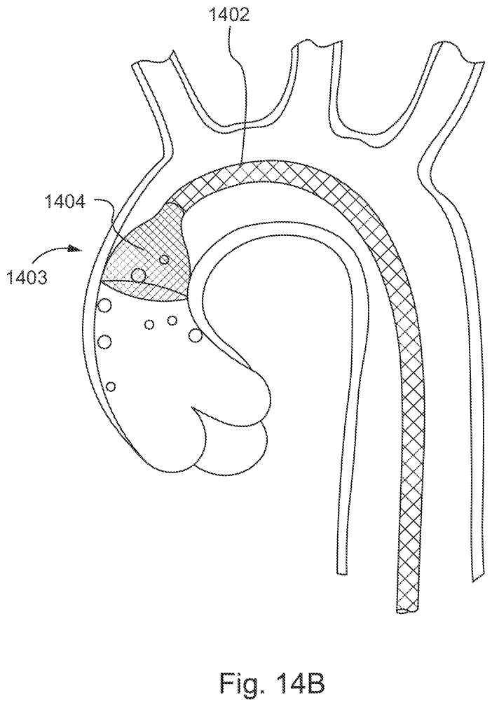

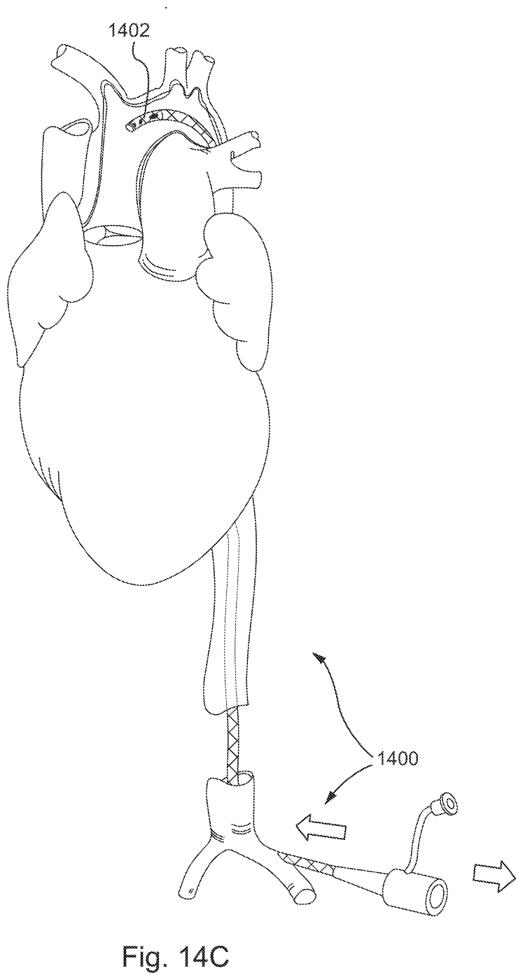

FIGS. 14A-C illustrate overviews of use of an embolic protection system 1400 according to some embodiments (e.g., see above), and also helps to illustrate methodology for performing an exemplary procedure (e.g., cardiac, e.g., valve repair or implantation). In such an example method, an/the embolic protection system 1400 is directed to a surgical location in a blood vessel or organ, via, entrance in a major blood vessel (e.g., femoral artery, carotid artery). Thereafter, a surgeon pulls or otherwise moves the tear-away (TA) sleeve in a proximal direction, tearing or otherwise separating the TA sleeve along at least a portion of its length beginning at the proximal end as the TA sleeve is pulled or otherwise moved in the proximal direction (resulting in the “accordion” condition 1401). The introducer sheath 1402 can then be pulled or otherwise directed in a proximal direction to expose a/the filter 1404 (which may also include a radiopaque edge marker 1405), such that, in some embodiments, as the filter is exposed, it expands (e.g., self-expansion), and the introducer sheath 1402 can fold-up upon itself in the proximal direction. In some embodiments, at least a portion of the filter 1402 and/or a distal section of the inner-body is configured to expand to a size to apply a force against an inner wall of the blood vessel or organ upon which the filter and/or distal section of the inner-body is positioned, so as to insure, for example, no flow or emboli can escape the system/filter. Moreover, in some embodiments, the method further includes recapturing the expanded filter 1404 by moving the introducer sheath 1402 distally upon completion of a procedure. The introducer sheath 1402 is pulled back to capture the expandable filter 1404 into the distal end of the introducer sheath as the sleeve is pushed forward, prior to removal of the filter 1404 from aorta 1403 (for example).

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that any and all parameters, dimensions, materials, and configurations described herein are meant to be an example and that the actual parameters, dimensions, materials, and/or configurations will depend upon the specific application or applications for which the inventive teachings disclosed herein is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are also directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Embodiments disclosed herein may also be combined with one or more features, as well as complete systems, devices and/or methods, to yield yet other embodiments and inventions. Moreover, some embodiments, may be distinguishable from the prior art by specifically lacking one and/or another feature disclosed in the particular prior art reference(s); i.e., claims to such embodiments are distinguishable from the prior art by including one or more negative limitations.

Also, various inventive concepts may be embodied as one or more methods, of which examples has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An embolic protection system (EPS) comprising:
an expandable filter including a plurality of pores sized to allow the flow of the blood with limited interruption and capture of emboli greater than the pore size;
an introducer sheath having a sheath diameter configured to accommodate at least the filter when unexpanded;
and
a tear-away (TA) sleeve having a sleeve diameter configured to accommodate the introducer sheath containing the expandable filter when unexpanded;
wherein:
the TA sleeve is configured to tear or otherwise separate along at least a portion of its length beginning at the proximal end as the TA sleeve is pulled or otherwise directed proximally prior to expansion of the expandable filter,
and
the expandable filter expands as it is distally exposed by the withdrawal of the introducer sheath in the proximal direction, such that a least a portion of the introducer sheath expands or contracts longitudinally.

2. The EPS of claim 1, wherein at least the expandable filter is configured for recapture after expansion by the distal end of introducer sheath upon distal movement of the introducer sheath.

3. The EPS of claim 2, wherein a proximal section of the introducer sheath expands or contracts longitudinally in an accordion-like fashion.

4. The EPS of claim 1, further comprising at least one of:
an expandable inner-body having a body diameter and a distal section; and
a dilator having a dilator diameter configured fit within the introducer sheath.

5. (canceled)

6. The EPS of claim 4, wherein the expandable filter is arranged on or adjacent at least the distal section of the inner-body.

7-10. (canceled)

11. The EPS of claim 4, wherein the sheath diameter is configured to accommodate the inner-body and the expandable filter, including the distal portion, when unexpanded.

12. The EPS of claim 1, wherein the introducer sheath includes at least one of: a wall having a thickness of between approximately 0.025 to 0.250 mm, an introducer hub arranged on a proximal end thereof, a flush port.

13. The EPS of claim 1, wherein the pores are sized between approximately 0.1 mm-1.0 mm.

14. The EPS of claim 1, wherein the introducer sheath includes at least one of: a wall having a thickness of between approximately 0.025 to 0.250 mm, an introducer hub arranged on a proximal end thereof, a flush port, and the pores are sized between approximately 0.1 mm-1.0 mm.

15. The EPS of claim 4, wherein:
the sleeve diameter of the TA sleeve is configured to accommodate the introducer sheath containing the inner-body and the expandable filter when unexpanded, and/or
the dilator includes at least one of a conical distal tip and a proximal dilator hub.

16-20. (canceled)

21. The EPS of claim 1, wherein the TA sleeve includes at least one pull-tab at a proximal end, or at least two pull tabs at the proximal end.

22. (canceled)

23. (canceled)

24. The EPS of claim 21, wherein the TA is configured to tear or otherwise separate along at least a portion of its length beginning at the proximal end at least initially via the at least one pull-tab as the TA sleeve is pulled or otherwise directed proximally prior to expansion of at least the expandable filter.

25. The EPS of claim 4, wherein at least a portion of the expandable filter and/or distal section of the inner-body is configured to expand to a size to apply a force against an inner wall of a blood vessel upon which the expandable filter and/or distal section of the inner-body is positioned.

26. The EPS of claim 1, wherein:
the entire length of the introducer sheath is configured to expand, or
at least a portion of the length of the introducer sheath is configured to expand.

27-29. (canceled)

30. The EPS of claim 1, further comprising a first housing configured substantially encase and/or seal a proximal end of the TA sleeve.

31. (canceled)

32. The EPS of claim 30, wherein the first housing comprises a guide lumen.

33. The EPS of claim 30, further comprising a second housing for encasing and/or sealing the first housing.

34. The EPS of claim 33, wherein the second housing comprises a guide lumen.

35-37. (canceled)

38. An embolic protection system (EPS) comprising:
an inner-body having an expandable body diameter and a distal section;
an expandable filter arranged on or adjacent at least the distal section of the inner-body, the expandable filter including a plurality of pores sized between approximately 0.100 mm-1.000 mm to allow the flow of the blood with limited interruption and capture of emboli greater than the pore size;
a radiopaque marker arranged on one or more areas of at least one of the expandable filter and distal section of the inner-body, the marker comprising at least one strand of at least one of platinum and gold included with the expandable filter, and/or a coating of at least one of platinum and gold;
an introducer sheath having a sheath diameter configured to accommodate the inner-body and expandable filter, including the distal portion, when unexpanded, wherein the introducer sheath includes:
a wall having a thickness of between approximately 0.025 mm to 0.250 mm,
an introducer hub arranged on a proximal end thereof, and
a flush port;
a tear-away (TA) sleeve having a sleeve diameter configured to accommodate the introducer sheath containing the inner-body and the expandable filter when unexpanded;
and
a dilator having a dilator diameter configured fit within the introducer sheath and within the body diameter, the dilator including a conical distal tip and a proximal dilator hub,
wherein:
the TA sleeve includes at least one pull-tab at a proximal end and is configured to tear or otherwise separate along at least a portion of its length beginning at the proximal end at least initially via the at least one pull-tab as the TA sleeve is pulled or otherwise directed proximally prior to expansion of at least the expandable filter,
at least the expandable filter expands as it is distally exposed by the withdrawal of the introducer sheath in the proximal direction, such that at least a portion of the introducer sheath expands or contracts an accordion-like fashion,
at least a portion of the expandable filter and/or distal section of the inner-body is configured to expand to a size to apply a force against an inner wall of a blood vessel upon which the expandable filter and/or distal section of the inner-body is positioned;
the introducer sheath is configured to expand over at least a portion of its length between approximately 10 cm and 30 cm;
and
at least the expandable filter is configured for recapture after expansion by the distal end of introducer sheath upon distal movement of the introducer sheath.

39. An embolic protection method (EPM) comprising:
optionally providing an embolic protection system (EPS) according to any of claims 1-34;
directing an/the EPS to a surgical location in a blood vessel or organ;
pulling or otherwise moving a/the tear-away (TA) sleeve in a proximal direction;
tearing or otherwise separating the TA sleeve along at least a portion of its length beginning at the proximal end as the TA sleeve is pulled or otherwise moved in the proximal direction,
pulling or otherwise directing an/the introducer sheath in a proximal direction to expose a/the expandable filter, such that:
as the expandable filter is exposed it expands, and
the introducer sheath expands or contracts longitudinally;
wherein at least a portion of the expandable filter and/or a distal section of the inner-body is configured to expand to a size to apply a force against an inner wall of the blood vessel or organ upon which the expandable filter and/or distal section of the inner-body is positioned;
and
optionally recapturing the expandable filter by moving the introducer sheath distally.

40-44. (canceled)
